(19) United States
(12) Patent Application Publication
(10) Pub. No.: US 2012/0107808 A1
(43) Pub. Date: May 3, 2012

(54) High throughput detection of gene-specific hydroxymethylation

(76) Inventors: Weiwei Li, Lindenhurst, NY (US); Jessica Li, Lindenhurst, NY (US)

(21) Appl. No.: 12/925,699

(22) Filed: Oct. 27, 2010

(51) Int. Cl. C12Q 1/68

(52) U.S. Cl. 435/6.11; 435/6.12; 435/6.19

(57) ABSTRACT

This invention provides a method for the detection of hydroxymethylation patterns in a DNA sample, especially in genetic regions. A test sample containing hydroxymethylated DNA is hybridized to capture oligonucleotides immobilized on a solid phase. The hydroxymethylated DNA in hybrid is detected using an antibody which specifically recognizes hydroxymethylcytosine structure the marker of DNA hydroxymethylation-followed by immuno-signal amplification. The present invention provides a method to detect gene-specific hydroxymethylation in a simple, rapid and high throughput format with high specificity and sensitivity.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

1. Field of the Invention

The invention provides a method for detecting hydroxymethylation patterns in a DNA sample, especially in genetic regions. A test sample containing hydroxymethylated DNA is hybridized to capture oligonucleotides immobilized on a solid phase. The hydroxymethylated DNA in the hybrid is detected using an antibody which specifically recognizes 5-hydroxymethylcytosine structure—the marker of DNA hydroxymethylation-followed by immuno-signal amplification. The present invention provides a′ method to detect gene-specific hydroxymethylation in a simple, rapid and high throughput format with high specificity and sensitivity.

2. Description of the Related Art

DNA methylation is an epigenetic modification which is catalyzed by DNA cytosine-5-methyltransferases (DNMTs) and occurs at the 5-position (C5) of the cytosine ring, within CpG dinucleotides. DNA methylation is essential in regulating gene expression in nearly all biological processes including development, growth, and differentiation (Laird P W et al: Annu Rew. Genet, 1996; Reik W et al: Science, 2001; Robertson K D et al: Nature Rew. Genet, 2005). Alterations in DNA methylation have been demonstrated to cause changes in gene expression. For example, hypermethylation leads to gene silencing or decreased gene expression while hypomethylation activates the genes or increases gene expression. Region-specific DNA methylation is mainly found in 5′-CpG-3′ dinucleotides within the promoters or in the first exon of genes, which is an important pathway for the repression of gene transcription in diseased cells.

Very recently, a novel modified nucleotide called 5-hydroxymethylcytosine (5-hmC) has been detected to be abundant in mouse brain and embryonic stem cells (Kriaucionis S et al: Science, 2009). 5-hydroxymethylcytosine was first>seen in bacteriophages in 1952 (Wyatt G R et al: Nature, 1952). In mammals, it can be generated by the oxidation of 5-methylcytosine, a reaction mediated by the Tet family of enzymes and Dnmt proteins (Tahiliani M et al: Science, 2009). 5-hmC is a hydroxylated and methylated form of cytosine. The 5-hydroxymethylcytosine structure may include 5-methylhydroxycytidine, 5-hydroxymethyl-2-deoxy-cytidine, 5-hydroxymethyl-2-deoxy-cytidne monophosphate (hmdCMP), 5-hydroxymethyl-2-deoxy-cytidne diphosphate (hmdCDP), and 5-hydroxymethyl-2-deoxy-cytidne triphosphate (hmdCTP). The broader functions of 5-hmC in epigenetics is still relatively unknown. However, a line of evidence showed that 5-hmC plays a role in DNA demethylation, chromatin remodeling and gene expression regulation, specifically in brain-specific gene regulation (Valinluck V et al: Cancer Res, 2007, Valinluck V et al: Nucleic Acid Res, 2004, Penn N W et al: Biochem J, 1976, Penn N W et al: Biochem J, 1972):
1) Conversion of 5-methylcytosine (5-mC) to 5-hmC greatly reduced the affinity of MBD proteins to methylated DNA;
2) Formation of 5-hmC by oxidative damage or by addition of aldehydes via Dnmts prevents Dnmt-mediated methylation of target cytosine.
3) 5-hmC may recruit specific binding proteins that alter chromatin structure or DNA methylation patterns.
4) 5-hmC is also specifically localized CpG regions
5) 5-hmC accounts for roughly 40 percent of the methylated cytosine in Purkinje cells and 10 percent in granule neurons.

Because of presence of 5-hmC in DNA probably with different function from 5-mC in gene regulation and discovery of the enzymes that produce 5-hmC, It is considered important to know the distribution of this new type of epigenetic DNA modification in different cell types and in different compartments of the genome of mammalians. It is more important to profile 5-hmC on a genome-wide scale and to determine the ratio of 5-hmC to 5-mC in different genetic regions. It is particularly important to identify gene-specific hydroxymethylation patterns in healthy and different disease states in human, not only for necessity of re-evaluating existing methylation datasets, but also for correctly recognizing the epigenetic regulation of physiological and pathological processes.

Several chromatography-based techniques including HPLC and TLC mass spectrometry are used for detecting 5-hmC (Kriaucionis S et al: Science, 2009; Penn N W et al: Biochem J, 1972). In chromatography-based analysis, DNA is digested into single nucleotides and the total genomic 5-hmC is quantified. However these methods are not able to identify gene-specific hydroxymethylation and are only suitable for the analysis of total 5-hmC content in a DNA sample. Furthermore these methods are labor intensive, time-consuming, or require large amounts of DNA (>250 ng) as the starting material for measurement, or rely on the use of expensive equipment. Currently used gene-specific methylation analysis methods including restriction enzyme digestion, bisulfite or MeDIP-mediated MS-PCR and sequencing are also demonstrated to be not suitable for 5-hmC or hydroxymethylated DNA detection at the gene level as 5-hmC and 5-mC are virtually indistinguishable with these methods (Huang Y et al: PLoS One, 2010; Jin S G et al: Nucleic Acid Res, 2010, Nestor C et al: BioTechniques, 2010). Thus, there are currently no methods available for detecting 5-hmC or hydroxymethylated DNA at the individual gene level and there is an ample need for establishing a method for the detection of gene-specific hydroxymethylation.

### BRIEF SUMMARY OF THE INVENTION

The present invention provides a simple and high throughput method that can rapidly detect and analyze hydroxymethylated patterns at the gene or promoter levels through immunodetection of 5-hmC structure followed by signal amplification. The method comprises the steps of:

1) Isolation and purification of DNA from biological materials;
2) Denaturation and fragmentation of DNA;
3) Immobilization of capture oligonucleotidse to a solid phase;
4) Hybridization of the fragmented DNA containing target sequence to capture oligonucleotide;
5) Detection of 5-hmC structure contained in the target DNA sequence with an anti-5-hmC specific antibody;
6) Detection of anti-5-hmC antibody with immuno-signal amplifiers;
7) Fluorescent or color development of immuno-signal amplifiers and quantification of fluorescent or color intensity.

Thus the invention allows for a rapid detection of gene-specific hydroxymethylation patterns to be carried out. The invention is based on the finding that the detection of 5-hmC located in the target DNA sequence can be quantitatively achieved through specific antibody recognition followed by immuno-signal amplification. Therefore the method presented in this invention significantly overcomes the weaknesses existing in prior technologies and enables gene-specific hydroxymethylation status to be detected rapidly and efficiently.

The method of the invention has the following advantages:

1. It detects gene hydroxymethylation by generating immuno-signal amplification of the hydroxymethylated DNA sequence, which would provide a simple, rapid, cost-effective and accurate method for routine use in analyzing hydroxymethylation patterns.

2. The signal amplification generated by the method of this invention is flexible and controllable. Amplification intensity can range from 100 fold to 5×10⁴ fold or greater, depending on the requirement.

3. It is able to process the detection assay in a solid phase format that is suitable for integration into microarrays or biochip platforms for high throughput analysis of gene-specific hydroxymethylation patterns.

### BRIEF DESCRIPTION OF THE DRAWINGS

FIG. 1 shows a diagram of the process for the detection of gene-specific hydroxymethylation. The process involves: (1) hybridization of the DNA fragment containing the target sequence to capture oligonucleotides immobilized on microplates or the microarray chips. Use of hydroxymethylated DNA oligonucleotides or polynucleotides containing a known number of 5-hmC as the control; (2) binding of anti-5-hmC antibody to the DNA fragment; (3) binding of nanobead signal amplifiers to anti-5-hmC antibody; (4) fluorescence or color development of nanobead signal amplifiers and signal intensity scanning; and (5) data analysis.

FIG. 2 shows the stability of nanobead signal amplifier bound to the target antibody.

FIG. 3 shows the sensitivity of the method of this invention in quantifying gene-specific hydroxymethylation.

FIG. 4 shows the specificity of the method of this invention in quantifying gene-specific hydroxymethylation patterns.

FIG. 5 shows the detection of gene-specific hydroxymethylation by the method of this invention using human tissue samples.

### DETAILED DESCRIPTION OF THE INVENTION

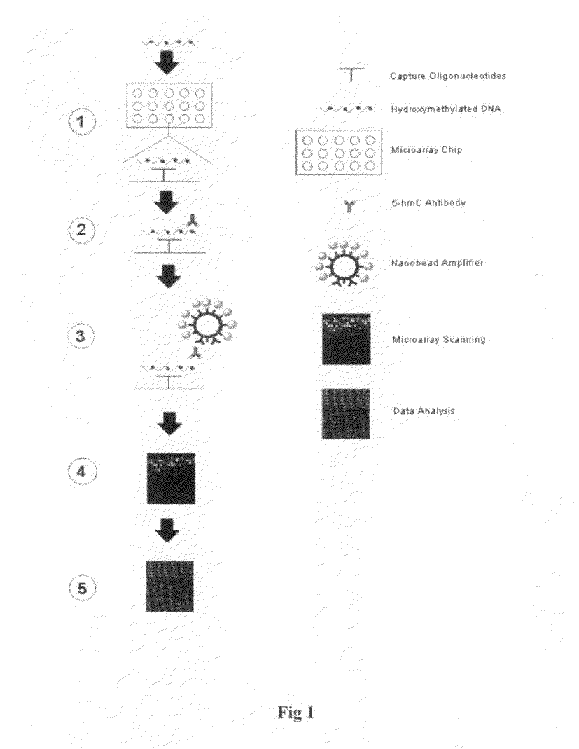

The present invention provides a method for high throughput detection of gene-specific hydroxymethylation by hybridizing DNA fragments containing the target DNA sequence to capture oligonucleotides immobilized on a solid phase followed by immunodetection of 5-hmC structure which is the marker of DNA hydroxymethylation. A basic outline of the method presented in this invention is described in FIG. 1. This method is particularly useful for identifying 5-hydroxymethylation status in a genetic region of interest in a short timeframe. This method is also particularly useful for a parallel determination of hydroxymethylation status of different genetic regions in a high throughput format.

According to the method of this invention, the single stranded oligonucleotides can be prepared by any conventional oligonucleotide preparation methods such as chemical synthesis and are immobilized as a capture oligonucleotide on a surface of solid phase such as tubes, microtiter plates, multi-well strips, films, beads, particles, papers, membranes and slides. The materials of solid phase include plastic, glass, metals, ceramics, polymers and so forth. The length of the single stranded oligonucleotides is 10-100 nucleotides, preferably 40-50 nucleotides with a sequence complementary to a DNA sequence containing at least 1 CpG site. The surface of solid phase is pre-coated with a substrate containing a reactive group. The oligonucleotides can be modified with a functional group that enables the oligonucleotides to covalently attach to a reactive group on the surface. For example, aminated oligonucleotides can be immobilized onto N-oxysuccinimide-coated glass slides (U.S. Pat. No. 6,391,655). Disulfide-modified oligonucleotides can be immobilized onto a mercaptosilanised glass surface by a thiol/disulfide exchange reaction (Rogers, Y H et al, Anal Biochem, 266: 23, 1999). Immobilization of oligonucleotides can also be achieved by physical absorption on poly-L-lysine, nitrocellulose, nylon membrane and polyacrylamide gels. The appropriate buffer and temperature are required for immobilizing such oligonucleotides on a solid phase by either chemical bonding or physical absorption.

According to the method of this invention, DNA could be isolated by lysis of cells with a lysis buffer containing a sodium salt, tris-HCl, EDTA, and detergents such as sodium dodecyl sulphate (SDS) or cetyltrimethylammonium bromide (CATB). Tissue fragments should be homogenized before lysing. For example, disaggregation of tissue fragments can be performed by stroking 10-50 times, depending on the tissue type, with a Dounce homogenizer. DNA can be further purified by mixing with a high concentration of sodium chloride and then adding it into a column pre-inserted with a silica gel, a silica membrane, or a silica filter. The DNA that binds to the silica matrix is washed by adding a washing buffer and is eluted with TE buffer or water. DNA can also be isolated and purified by using commercially available DNA extraction kits such as QiaAmp blood or tissue kits (Qiagen). The starting materials for DNA extraction can be from various species including, but not limited to, fresh tissues, frozen tissues, formalin fixed and paraffin embedded tissues, body fluids, and cultured cells.

DNA can be mechanically sheared, chemically sheared, or enzymatically digested to yield an appropriate length of the DNA fragment. Usually, 200-500 by of sheared or digested DNA is required for hybridization with capture oligonucleotides immobilized on a solid phase. Mechanical shearing of DNA can be performed by nebulization or sonication, preferably sonication using a sonicator processor such as the EpiSonic processor (Epigentek). Chemical shearing can be performed by heating, acid catalytic hydrolysis, alkaline catalytic hydrolysis, hydrolysis by metal ions, or hydroxyl radicals. Enzymatic digestion of DNA can be performed by using a variety of restriction enzymes, preferably by using DNAse I to facilitate the subsequent hybridization step.

The sheared DNA fragments are denatured by heating at 95° C. to 99° C. for an appropriate time and then hybridized to the capture oligonucleotides immobilized on the surface of the solid phase. The sequence of the capture oligonucleotides is complementary to a portion of the nucleotide sequence of a target DNA fragment. The quick hybridization (0.5-1 h) can be achieved by increasing the hybridization temperature and/or salt concentration of the hybridization solution. After the capture of the target DNA sequence, the hybridization solution is removed and the surface of the solid support is washed. A polyclonal or monoclonal antibody specific to 5-hmC structure, as a capture antibody, is added to bind to 5-hmC contained in the DNA fragments. After incubation and washing, a solution containing signal amplifiers is added at an appropriate concentration and binds to an anti-5-hmC antibody. After binding of signal amplifiers to the capture antibody, the surface of the solid phase is washed again and through such a way, a capture antibody molecule can bind at least a signal amplifier.

A signal amplifier can be an affinity antibody or ligand specific to the capture antibody, which conjugated with a variety of labeling moieties such as fluorescent or color dyes. The signal amplifier can also be a DNA dendrimer conjugated with the affinity antibody and a variety of labeling moieties, or tyramide-mediated catalyzed reporter, or an ADHP (Amplex red)-mediated hydrogen peroxide/peroxidase system. Preferably, the signal amplifier is nanobead amplifier that is consisted of a carrier bead, an affinity antibody or a ligand specific to the capture antibody, and labeling moieties. The carrier bead includes but is not limited to polypropylene bead, polystyrene bead, glass bead, metal bead, silica bead, latex bead, and magnetic bead. The bead size may be from 10 nanometers to 1000 nanometers in diameter, preferably from 20 nanometers to 500 nanometers, more preferably from 50 nanometers to 300 micrometers, most preferably from 100 nanometers to 200 micrometers. Most of the carrier beads can be available commercially such as Adembead from Ademtech. The labeling moieties, depending on the requirement of the assay, include but are not limited to horse radish peroxidase (HRP), alkaline phosphotase (AP), biotin, fluorescein (FITC), Cy3, Cy5, rhodamine, texas red, Alexa fluor, BODIPY, phycoerythrin, captivate ferrofluid, cascade blue, marine blue, Oregon green, pacific blue, and quantum dot.

The preparation of the nanobead amplifier can be accomplished through immobilizing an affinity antibody or ligand and labeling moieties to the carrier bead. For example, the affinity antibody or ligand and the labeling moieties can be first conjugated with biotin and then simultaneously coupled to a streptavidin-coated bead. The number of the coupled affinity antibody molecules or ligand and labeling moieties is dependent on the size of the carrier bead. An appropriate ratio of immobilized affinity antibody or ligand to labeling moieties can be from 1:10 to 1:10,000, preferably 1:200 to 1:1,000. A 20 nm bead can bind approximately 1-2 affinity antibodies and 20-200 labeling moieties, depending on the size of labeling moieties. A 200 nm bead is able to allow approximate 20 affinity antibodies and 2,000-20,000 labeling moieties to be immobilized.

A polyclonal or monoclonal antibody which recognizes and binds to 5-hmC can be generated according to various methods described in the prior art. For example, the 5-hmC polyclonal antibody can be generated by using the Abgent protocol: (1) Preparation of 5-hmC-KLH conjugates. KLH may be, modified with 3-sulfo-N-hydroxysuccinimide ester sodium salt before conjugation. The conjugates of KLH-5-hmC can be identified by ultraviolet spectrophotometer; (2) Injection of KLH-5hmC into rabbits. Injections of the antigen are given in multiple sites to stimulate the best immunity. The rabbits are boosted at 21 day intervals until peak antibody titers are reached (6-8 re-immunizations); (3) Blood sample collection. Blood is collected from the central ear artery and allowed to clot and retract at 37° C. overnight. The clotted blood is then refrigerated for 24 hours before the serum is decanted and clarified by centrifugation; and (4) ELISA test of antibody titers and affinity purification.

DNA oligonucleotides or polynucleotides containing the known number of 5-hmC at CpG sites can be used as the positive control. The positive control can be synthesized commercially or generated by PCR amplification. For example, PCR fragments containing 5-hydroxymethylated cytosine with a length of 60-300 by can be generated using human MLH1 promoter derived sequences by incorporating dhmCTP (5-hydroxy-methylcytidine) with dATP, dGTP, and dTTP. PCR fragments containing 5-methylcytosine with the same length of 60-300 by can be generated using human MLH1 promoter derived sequences by incorporating dmCTP (5-methylcytidine) with dATP, dGTP, and dTTP. PCR fragments containing unmethylated cytosine with same length of 60-300 by can be generated using human MLH1 promoter derived sequences by incorporating dCTP (cytidine) with dATP, dGTP, and dTTP.

According to the method of this invention, as low as a single DNA molecule containing only two to three 5-hmC can be detected through fluorescent measurement. The detection of the gene-specific hydroxymethylation patterns can be carried out in a single strip well, or a multiple well strip, or a 96 to 1536 well microplate or a microchip slide. For detecting gene-specific hydroxymethylation in high density microchip format, human CpG island microarray chips, which contain 237,000 capture probes covering 27,800 CpG islands can be purchased from Agilent Technologies and used for the testing. Fragmented and denatured DNA can be hybridized to the chips. After hybridization, a 5-hmC antibody, and then the nanobead amplifer immobilized with fluorescent moieties such as Cy5 or phycoerythrin is in turn applied to the chip and the fluorescent measurement is carried out with a GenePix 4000B microarray scanner. A complete detection or quantification of the gene-specific hydroxymethylation patterns needs only 4 hours.

The method of this invention is useful in detecting gene-specific hydroxymethylation patterns using a biological sample. The method of this invention may be particularly useful in detecting gene-specific hydroxymethylation patterns in a clinical sample with a minute amount of DNA. These clinical samples may include but are not limited to tissue biopsy, tissue section, formalin fixed paraffin embedded (FFPE) specimens, plasma, serum, cerebro-spinal fluid, tears, sweat, lymph fluid, saliva, nasal swab or nasal aspirate, sputum, bronchoalveolar lavage, breast aspirate, pleural effusion, peritoneal fluid, glandular fluid, amniotic fluid, cervical swab or vaginal fluid, ejaculate, semen, prostate fluid, urine, conjunctival fluid, duodenal juice, pancreatic juice, bile and stool. The method of this invention may be more particularly useful in the development of a simple and cost-effective assay for detecting DNA hydroxymethylation biomarkers of important diseases such as cancer, infectious diseases, immune diseases, and neurodegenerative diseases.

The method of this invention for detecting gene-specific hydroxymethylation is further illustrated in the following examples:

### Example 1

The experiment was carried out to test the stability and the efficiency of the nanobead amplifer binding to the capture antibody.

1. Preparation of nanobead amplifier. The nanobead size and type are selected based on that the specific binding of the beads to the target should be stable and tight with minimal non-specific background, while the surface area of the beads should be as large as possible for maximally conjugating affinity antibody and labeling moieties. The streptavidin-coated magnetic beads in a diameter of 200 nm were found to be the most appropriate. To prepare the functionalized nanobead amplifier, 1 mg of the beads (approximately 1×10¹⁰ beads) was washed twice with PBS and resuspened in the 1 ml of PBS. 20 μl (10 μg/ml) of biotin-labeled anti-rabbit IgG (Pierce) as affinity antibody and 20 μl (50 pmol/μl) of biotin-labeled HRP (Sigma) as labeling moieties are added into the suspended beads solution, respectively. The mixed solution was incubated at room temperature for 1 h and then washed 4 times with PBS by applying a magnetic field. The bead pellet was then suspended in the 1 ml PBS and stored at 4° C. Through such a way, one bead could conjugate approximately 20 affinity antibody molecules and 5,000 labeling moieties.

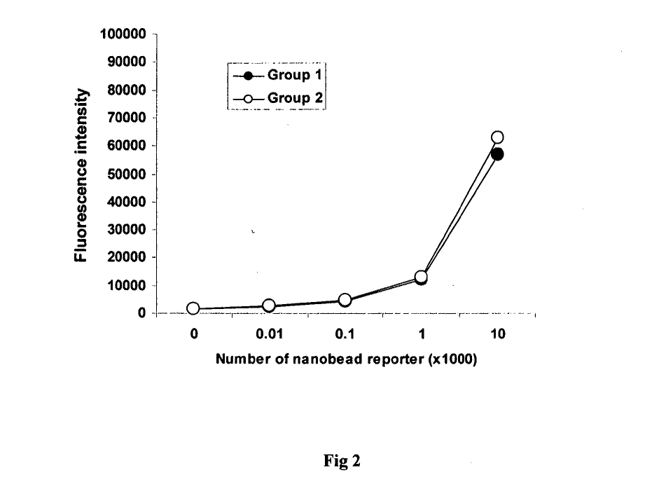

2. Testing the stability and the efficiency of the nanobead amplifier binding to capture antibody. In Group1, nanobead amplifiers were diluted to different concentrations and 100 μl of the diluted nanobead amplifier solutions were added into the 5-hmC polyclonal antibody coated stripwells. The wells were washed with PBS-T for 6 times after 1 h incubation. In Group 2, the same amounts of diluted nanobead amplifier solutions were added into 0.5 ml vials. The fluorescence development was carried out for both groups by adding an ADHP/hydrogen peroxide solution and the fluorescent intensity was measured at 530ex/580em nm using a fluorescence microplate reader. The nanobead amplifier in a diameter of 200 nm was able to stably bind to the target antibody after extensively washing (FIG. 2). Approximately 100% of nanobead amplifiers added into wells bound to the target antibody.

### Example 2

This experiment was carried out to test the sensitivity of the method of this invention in quantifying gene-specific hydroxymethylation.

100 μl (2 μM) of a 54 mer aminated capture oligonucletides complementary to the sequences within the promoter/exon1 region of gene MLH1, were immobilized to NOS-DNA Bind stripwells (Corning). The wells without oligonucleotides were used as the blank. After washing with 0.1 M of carbonate buffer, the stripwells were dried and used for hybridization. PCR fragments containing four 5-hydroxymethylcytosines were mixed with DNA isolated from a HCT116 colon cancer cell line that contains little to no 5-hmC and has no MLH1 methylation. The ratios of hydroxymethylated fragments to HCT116 DNA were 0.005, 0.01, 0.1, 0.5, 1, 5, and 10 pg to 100 ng of HCT116 DNA. 100 μl of mixed DNA (100 ng/100 μl) was denatured by boiling for 5 min and then hybridized to the strip wells immobilized with capture oligonucleotides. The PCR fragments containing unmethylated cytosine were also mixed with HCT116 DNA and used as the negative control. Fast hybridization was performed at 65° C. for 1 h by using a rapid hybridization solution containing 10 mM Tris-HCl, 0.5 M NaCl and 0.2 M MgCL2. Following hybridization, the wells were washed for 3 times with a PBS-T wash buffer and blocked for 30 min with a block buffer (2% BSA).

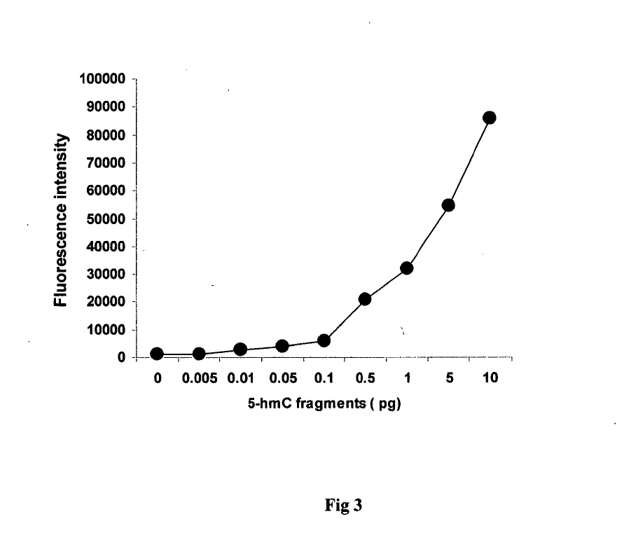

After the block buffer was removed, 100 μl of 5-hmC polyclonal antibody at concentration of 1 μg/ml was added into the wells to recognize and bind to 5-hmC. After 1 h incubation, the antibody buffer is removed and the wells were washed with PBS-T for 3 times. The nanobead amplifier solution was diluted with PBS to 1×10⁶/ml, and 100 μl of the diluted nanobead amplifier were added and incubated for 30 min at room temperature. The nanobead amplifier solution was then removed and washed with PBS-T for 3-4 times. The fluorescence development was carried out by adding an ADHP/hydrogen peroxide solution and the fluorescent intensity was measured at 530ex/580em nm using a fluorescence microplate reader. As shown in the FIG. 3, sequence-specific hydroxymethylation can be detected from DNA containing as low as 0.01 pg of hydroxymethylated PCR fragments.

### Example 3

The experiment was carried out to examine the specificity of the method based on this invention in detecting gene-specific hydroxymethylation patterns

100 μl (2 μM) of a 54 mer aminated capture oligonucletides complementary to the sequences within the promoter/exon1 region of gene MLH1, were immobilized to NOS-DNA Bind stripwells (Corning). The wells without oligonucleotides were used as the blank. After washing with 0.1 M of carbonate buffer, the stripwells are dried and used for hybridization. In Group 1, PCR fragments containing four 5-hydroxymethylcytosines were mixed with DNA isolated from HCT116 colon cancer cell line. The ratios of hydroxymethylated fragments to HCT116 DNA are 0.01, 0.1, 0.5, 1, 5, 10, and 50 pg to 100 ng of HCT-116 DNA. 100 μl of mixed DNA (100 ng/100 μl) was denatured by boiling for 5 min and then hybridized to the strip wells immobilized with capture oligonucleotides. In Group 2, PCR fragments containing four 5-methylcytosines were mixed with DNA isolated from HCT116 colon cancer cell line. The ratios of methylated fragments to HCT116 DNA are 0.1, 0.5, 1, 5, 10, 50, and 100 pg to 100 ng of HCT-116 DNA. 100 μl of mixed DNA (100 ng/100 μl) was denatured by boiling for 5 min and then hybridized to the strip wells immobilized with capture oligonucleotides. The PCR fragments containing unmethylated 5-cytosine were also mixed with HCT116 DNA and used as the negative control. Fast hybridization was performed using rapid hybridization solution at 65° C. for 1 h. Following hybridization, the wells were washed for 3 times with PBS-T wash buffer and blocked for 30 min with block buffer (2% BSA).

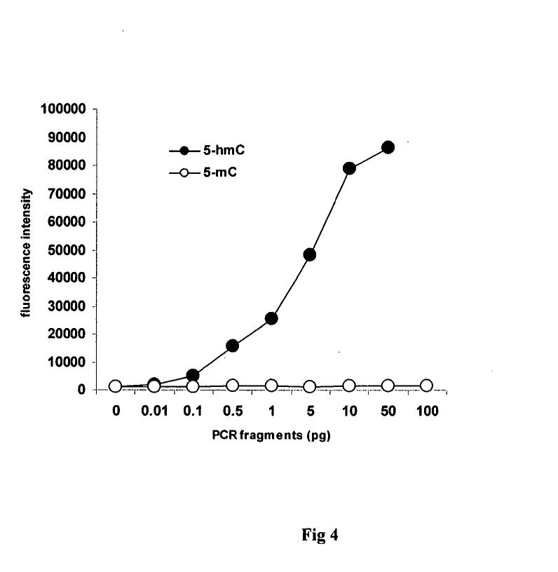

After the block buffer was removed, 100 μl of 5-hmC polyclonal antibody at concentration of 1 μg/ml were added into the wells to recognize and bind to 5-hmC or 5-mC, respectively. After 1 h incubation, the antibody buffer was removed. The wells were then washed with PBS-T for 3 times. The nanobead amplifier solution was diluted with PBS to 1×10⁶/ml, and 100 μl of the diluted nanobead amplifier were added and incubated for 30 min at room temperature. The nanobead amplifier solution was then removed and washed with PBS-T for 3-4 times. The fluorescence development was carried out by adding ADHP/hydrogen peroxide solution and fluorescent intensity was measured at 530ex/580 em nm using a fluorescence microplate reader. As shown in the FIG. 4, only sequence-specific hydroxymethylation was detected from DNA containing 5-hmC or 5-hmC fragments.

### Example 4

The experiment was carried out to examine the ability in detecting gene-specific hydroxymethylation in human tissue or cell samples.

100 μl (5 μM) of a 54 mer aminated capture oligonucleotides complementary to the sequences within the promoter/exon1 region of gene MLH1, and complementary to the sequences within the promoter/exon1 region of gene RASSF1A were immobilized to NOS-DNA Bind stripwells. The wells without oligonucleotides were used as the blank. After washing with 0.1 M of carbonate buffer, the stripwells were dried and used for hybridization. 500 ng of DNA isolated from human brain, kidney, colon, Hela cervical cancer cell line and HCT116 colon cell line were fragmented to 200-600 by by sonication with the Episonic™ processor (Epigentek), denatured by boiling for 5 min and then hybridized to the strip wells immobilized with capture oligonucleotides. The PCR fragments containing 5-hmC or only unmethylated cytosine were used as the positive control and negative control, respectively. Fast hybridization was performed at 65° C. for 1 h by using a rapid hybridization solution. Following hybridization, the wells were washed for 3 times with a PBS-T wash buffer and blocked for 30 min with a block buffer (2% BSA).

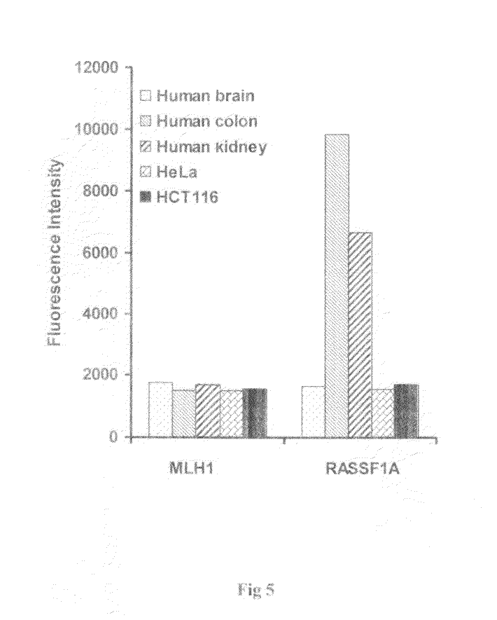

After the block buffer is removed, 100 μl of 5-hmC polyclonal antibody at concentration of 1 μg/ml were added into the wells to recognize and bind to 5-hmC. After 1 h incubation, the antibody buffer was removed and the wells were washed with PBS-T for 3 times. The nanobead amplifier solution was diluted with PBS to 1×10⁶/ml, and 100 μl of the diluted nanobead amplifier were added and incubated for 30 min at room temperature. The nanobead amplifier solution was then removed and washed with PBS-T for 3-4 times. The fluorescence development was carried out by adding ADHP/hydrogen peroxide solution and fluorescent intensity was measured at 530ex/580em nm using a fluorescence microplate reader. As shown in the FIG. 5, RASSF1A hydroxymethylation can be detected in human kidney and colon tissues.

What is claimed is:

1. A method for the detection of the presence or absence of a hydroxymethylated DNA sequence in a DNA sample through immuno-affinity signal amplification of said hydroxymethylated DNA sequence comprising steps of: (a) a capture probe consisting of a nucleotide sequence complementary to a nucleotide sequence corresponding to said hydroxymethylated DNA sequence; (b) immobilization of said capture probe to a solid phase; (c) hybridization of said DNA sample to said capture probe; (d) addition of an anti-5-hydroxymethylcytosine structure antibody that reacts with 5-hydroxymethylcytosine contained in hydroxymethylated DNA sequence hybridized to said capture probe; (e) binding of a nanobead amplifier consisting of a carrier bead, an affinity antibody and labeling moieties, to an anti-5-hydroxymethylcytosine antibody; and (f) detection of signal intensity generated from said nanobead amplifier wherein the signal intensity of said nanobead amplifier is indicative of the presence of hydroxymethylated DNA sequence in the sample DNA.

2. The method according to claim 1 wherein said capture probe is a single stranded oligonucleotide containing at least 1 CpG site with a length of 100 nucleotides or less.

3. The method according to claim 1 wherein said carrier bead is a polypropylene bead, or a polystyrene bead, or a glass bead, or a metal bead, or a silica bead, or a magnetic bead with size from 5 nm to 900 nm in diameter.

4. The method according to claim 1 wherein said carrier bead is coated with streptavidin, avidin or neutravidin.

5. The method according to claim 1 wherein said an affinity antibody is labeled with biotin and is anti-mouse, or anti-rabbit, or anti-goat or anti-sheep or anti-chicken IgG or IgM and labeled with biotin.

6. The method according to claim 1 wherein said the labeling moieties are selected from horse radish peroxidase (HRP), alkaline phosphotase (AP), fluorescein (FITC), Cy3, Cy5, rhodamine, dynabeads, texas red, Alexa fluor, BODIPY, phycoerythrin, and quantum dot.

7. The method according to claim 1 wherein said the labeling moiety is HRP.

8. The method according to claim 1 wherein said the labeling moiety is Alexa fluor.

9. The method according to claim 1 wherein said the labeling moiety is AP.

10. The method according to claim 1 wherein said the labeling moieties are Cy3 and Cy5.

11. The method according to claim 1 where in said affinity antibody and labeling moieties are immobilized to the carrier bead at a ratio of 1:10 to 1:1,000.

12. The method according to claim 1 wherein said 5-hydroxymethylcytosine structure is 5-hydroxymethylcytosine, or 5-hydroxymethylcytidine, or 5-hydroxymethyldeoxycytidine.

13. The method according to claim 1 wherein said 5-hydroxymethylcytosine structure antibody is selected from mouse monoclonal anti-5-hydroxymethylcytosine, mouse monoclonal anti-5-hydroxymethylcytidine, rat monoclonal anti-5-hydroxymethylcytosine, rabbit polyclonal anti-5-hydroxymethylcytidine, goat polyclonal anti-5-hydroxymethylcytosine, sheep polyclonal anti-5-hydroxymethylcytidine, or recombinant ScFv anti-5-hydroxymethylcytosine.

14. The method according to claim 1 wherein said 5-hydroxymethylcytosine structure antibody is rabbit polyclonal anti-5-hydroxymethylcytosine.

15. The method according to claim 1 wherein said 5-hydroxymethylcytosine structure antibody is mouse monoclonal anti-5-hydroxymethylcytosine.

16. The method according to claim 1 wherein said solid phase is a multi-well plate.

17. The method according to claim 1 wherein said solid phase is a microscope slide.

18. The method according to claim 1 wherein said solid phase is a microchip.

19. The method according to claim 1 wherein said solid phase is a nitrocellulose membrane.

20. The method according to claim 1 wherein said DNA sample is from tissues or cells of mammalian origin, or eukaryotic origin, or plant origin.
